(19) United States
(12) Patent Application Publication
(10) Pub. No.: US 2012/0022333 A1
(43) Pub. Date: Jan. 26, 2012

(54) APPARATUS AND SYSTEM FOR SIMULTANEOUS USE OF MULTIPLE INSTRUMENTS

(75) Inventors: David Main, Leeds Yorkshire (GB); Peter Moran, Leeds Yorkshire (GB)

(73) Assignee: SURGICAL INNOVATIONS LIMITED, Leeds Yorkshire (GB)

(21) Appl. No.: 13/260,830

(22) PCT Filed: Mar. 29, 2010
(86) PCT No.: PCT/GB10/50524

§ 371 Date: Sep. 28, 2011

(30) Foreign Application Priority Data
Apr. 1, 2009 (GB) 0905603.7

(51) Int. Cl. A61B 1/32

(52) U.S. Cl. 600/201

(57) ABSTRACT

An apparatus for allowing simultaneous use of at least two surgical instruments through a single incision. The apparatus includes a first part having a proximal end, a distal end, an inner surface and an outer surface. The outer surface engages an incision and holds the incision in an open position. The inner surface defines a passageway through the first part from the proximal to the distal ends. The apparatus also includes a second part attached to the proximal end of the first part for sealing the proximal end of the first part and having at least two ports for receiving a surgical instrument. At least one of the ports includes a ball joint to allow articulation of the port relative to the second part. The ball joint allows the orientation of the port and the surgical instrument within it to be moved easily relative to the apparatus.

The present invention relates to an apparatus for allowing simultaneous use of two or more surgical instruments through a single incision and a system comprising such an apparatus. The present invention is particularly applied to laparoscopic surgical procedures.

Conventional laparoscopic surgical procedures typically involve several incisions for the various instruments used during the procedure. Although the incisions are much smaller than open surgery, the presence of several incisions can result in increased recovery times and discomfort for the patient after the procedure. Typically there will be four incisions which results in multiple scarring.

Recently, it has been proposed to carry out laparoscopic procedures through a single incision, which has the advantage of less scarring. These procedures are generally known as single incision laparoscopic surgery. These systems still require the use of several instruments simultaneously. Consequently, systems have been proposed which allow the use of several instruments simultaneously through a single incision.

One system that has been proposed is marketed as “SILS port” by Covidien AG. The SILS port comprises a relatively flexible port defining three channels from its proximal to distal end. In use, the SILS port is compressed and inserted into an incision. Its natural elasticity then returns it to its original shape where it holds the incision open and provides sealing. Valve ports for receiving surgical instruments are then inserted into each of the channels.

The SILS port has some disadvantages. The material forming the SILS port must have natural elasticity to return to its original shape and then hold the incision open and provide sealing. In practice this means that the material must have a relatively high Young's modulus. This reduces the ability to move the instrument relative to the SILS port. When a surgeon desires to move an instrument to a different angle relative to the SILS port, it is thought that the majority of the movement will in fact be accommodated by movement of the port itself because the surrounding tissue is likely to have a lower effective elasticity than the port. This can exert undesirable forces on the incision and possibly cause harm to the patient. In addition, if it is desired to move two instruments simultaneously in different directions, the high Young's modulus makes his difficult and reduced the range of relative movement. Another disadvantage is that the limited deformation available because of the high Young's modulus may mean that the port deforms slightly but not enough to guarantee good sealing between the port and the instrument.

An alternative system is marketed by Advanced Surgical Concepts Limited under the names Triport and Quadport. The Triport and Quadport comprise distal and proximal rings joined with a flexible membrane. The distal ring is inserted through the incision and then retracted so that it rests against the internal wall of the incision, with the proximal ring against the outer wall. The flexible membrane then holds the incision open. Connected to the outer proximal ring is a boot section which contains three or four ports for receiving surgical instruments. If it is desired to alter the orientation of an instrument, the majority of the movement is accommodated by moving the position of the instrument relative to the port. This limits the range of movement available and introduces possible problems with sealing because the seal must be operative over a wide range of relative orientations of the instrument to the port.

It would be desirable to provide an improved port system for use with single incision surgery. Accordingly, the present invention provides an apparatus comprising at least two ports for receiving surgical instruments. At least one of the ports is connected via a ball joint so that a surgical instrument within the port can be moved relative to the apparatus through a wide range of relative orientations without compromising sealing.

According to an aspect of the present invention, there is provided an apparatus for allowing simultaneous use of at least two surgical instruments through a single incision, the apparatus comprising:
a first part having a proximal end, a distal end, an inner surface and an outer surface, wherein the outer surface is for engaging an incision and holding the incision in an open position and the inner surface defines a passageway through the first part from the proximal to the distal ends; and
a second part attached to the proximal end of the first part for sealing the proximal end of the first part and comprising at least two ports for receiving a surgical instrument, and wherein at least one of the ports comprises a ball joint to allow articulation of the port relative to the second part.

The use of a ball joint allows the orientation of the port and the surgical instrument within it to be moved easily relative to the apparatus. This means that the apparatus can remain in place without placing undue strain on the incision and the freedom of movement of the port during surgery is less restricted. Another advantage is that if more one port comprises a ball joint, instruments in different ports can be moved in opposite directions relative to each other simultaneously more easily.

Preferably, each port further comprises a valve for sealing the surgical instrument and if the port comprises a ball joint, the valve is positioned separate from the ball joint. The use of a ball joint allows the valve for sealing the surgical instrument to be positioned away from the ball joint. For example, in one embodiment the valve may be located in a neck which extends proximally from the ball joint. This means that the valve can remain in a relatively constant position relative to the surgical instrument, no matter what the position of the ball joint. Thus, the design of the valve can be optimised to provide good sealing in a particular relative orientation allowing a simpler construction and more effective sealing. Any suitable valve may be used, for example a non-return valve with lip seal.

In one embodiment, the ball joints may comprise sealing fins located in the socket. Although a ball joint itself may provide effective sealing, dependent on the level of contact between the ball and the socket, fins can also be moulded within the socket to provide sealing. The use of fins may allow the ball joint to move with less friction while still maintaining good sealing.

Preferably, the second part is releasably attached to the first part. This enables the second part to be varied depending on use. For example, a variety of second parts may be provided with different numbers or configurations of ports. It also allows the second part to be easily made reusable. In alternate embodiments either or both of the first and second parts may be reusable.

When the second part is releasably attached to the first part, the first part can be inserted into the incision and the second part attached after insertion. This allows simple, reliable insertion of the first part. For example, in one embodiment the first part may be adapted to be inserted into an incision using a trocar. The trocar can open the incision gradually, for example by including a conical end portion, and ensure that the first part is installed securely. A trocar can then be removed before attaching the second part.

In an alternative embodiment, the second part may be attached to the first part using a hinge or other form of pivotal connection. This still allows insertion of the first part using a trocar, the second part can simply be pivoted to one side during insertion.

According to another aspect of the invention, a system is provided including a trocar and an apparatus with a first part adapted for insertion into an incision using a trocar as described above.

Embodiments of the invention will now be described by way of example with reference to the accompanying drawings, in which:

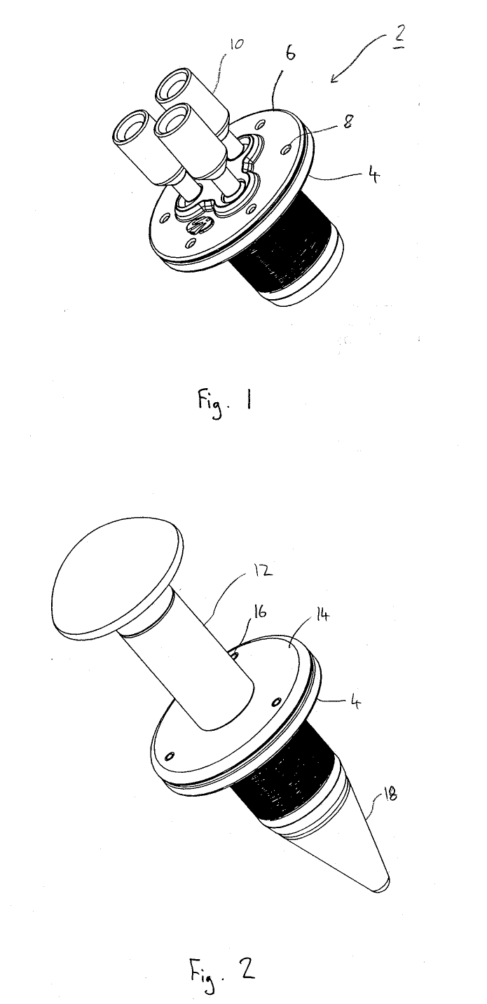

FIG. 1 depicts a perspective view of an apparatus according to a first embodiment of the invention;

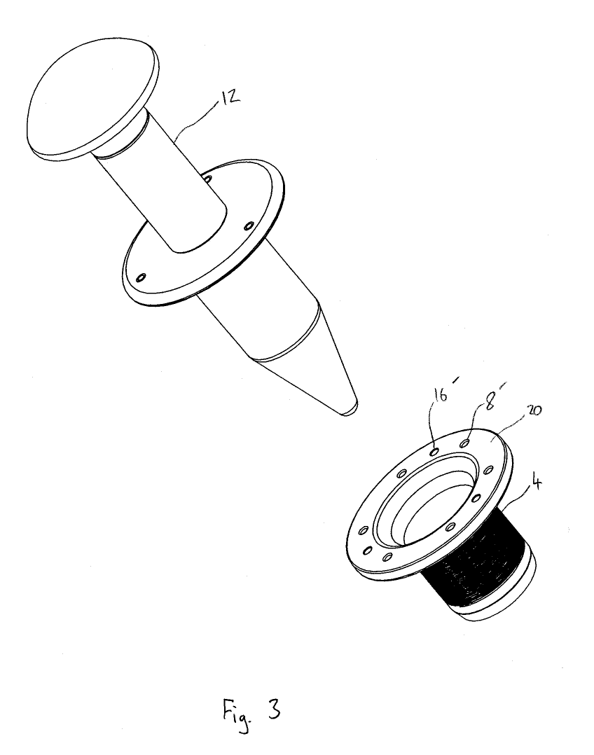

FIG. 2 depicts a perspective view of a first part of the embodiment of FIG. 1 connected to a trocar for insertion;

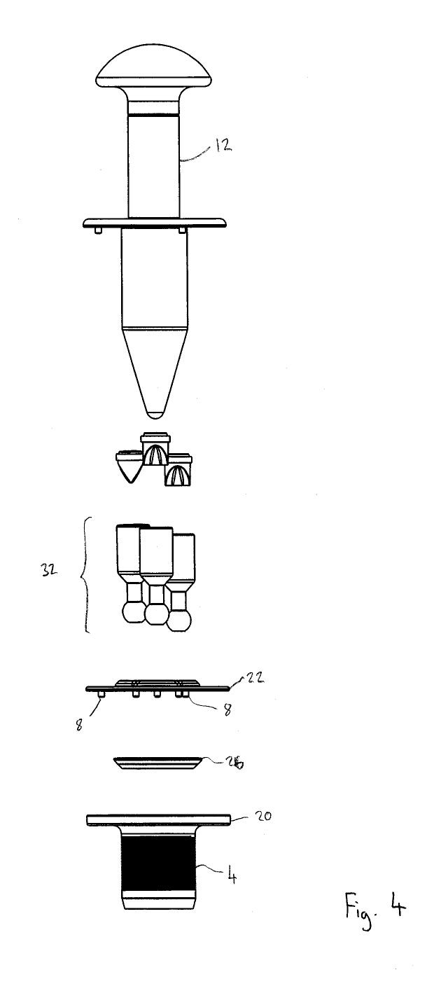

FIG. 3 depicts an exploded view of the assembly of FIG. 2;

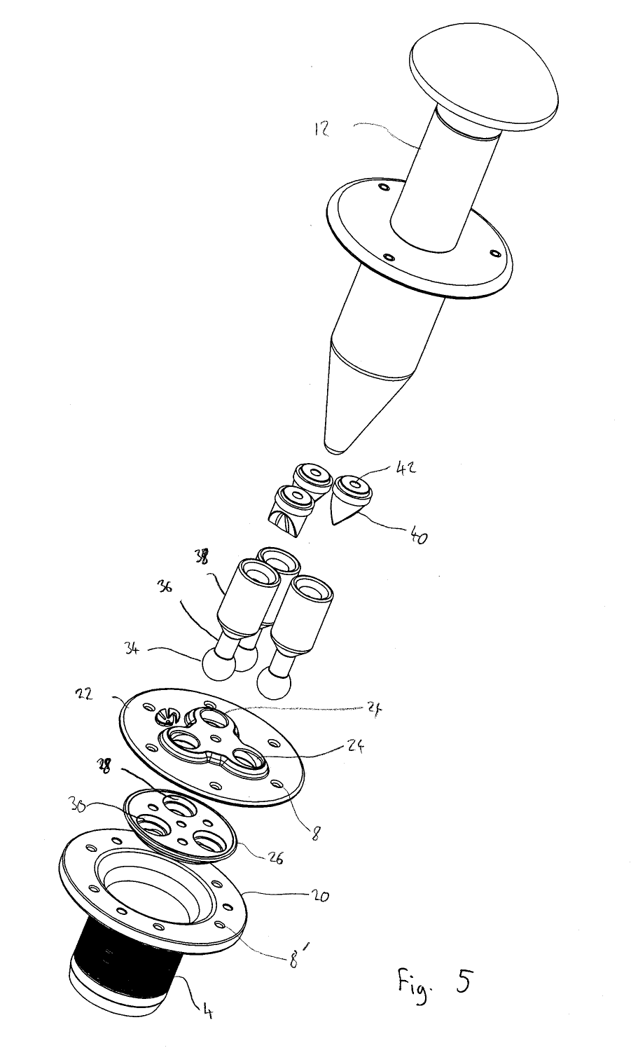

FIG. 4 is an exploded side view of all the components of a system according to the present invention;

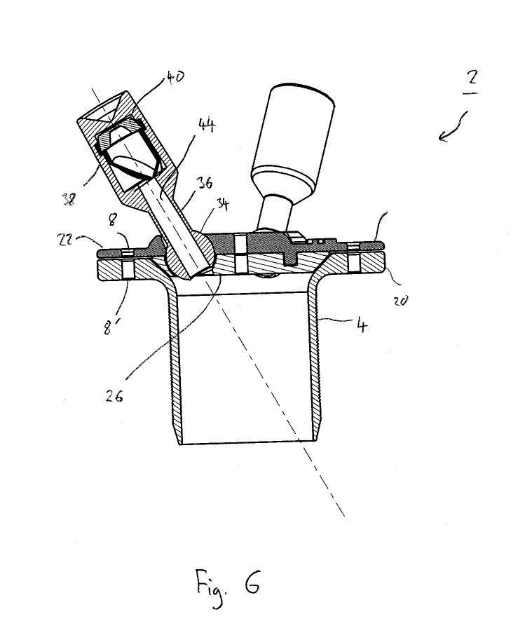

FIG. 5 depicts an exploded perspective view of a system according to the present invention; and

FIG. 6 depicts a cross-section through an apparatus according to the present invention.

FIG. 1 depicts a perspective view of an apparatus 2 for allowing simultaneous access of more than one surgical instrument through a single incision. The apparatus 2 comprises a first part 4 with a second part 6 attached at its proximal end. The second part 6 is attached via connection points 8. In this embodiment there are six connection points 8 evenly spaced around the periphery of the first part 4 and the second part 6. The connection points 8 can be simple pressed fit attachments, for example, a projection on one part engaging a recess on another. In alternate embodiments the connection points 8 may be configured to receive screws or another form of separate fastening.

Extending from the second part 6 are three ports 10. In use the ports 10 receive a surgical instrument. To allow the ports 10 to pivot relative to the apparatus 2 (along with any surgical instrument received in them) a ball joint is provided at the proximal end of the ports 10, at their connection to the second part 6. (The ball joint will be described in more detail below).

FIG. 2 depicts a perspective view of a first part 4 attached to a trocar 12 for delivery. The trocar 12 has a plate 14 with connection points 16 for engaging corresponding connection points on the first part 4. As with the connection between the first part 4 and the second part 6, the connection point 16 could comprise a protrusion that engages a recess or some other form of connection, such as a screw or fastener.

The trocar comprises a handle section towards its proximal end and a rounded, generally conical distal section 18. In use, the trocar is used to insert the first part 4 into an incision. The distal end of the trocar is inserted first. The rounded end and conical portion 18 then assist in enlarging the incision while reducing the risk of tearing and damaging the tissue around the incision. Further insertion of the trocar 12, once the incision has been opened by the conical section 18 results in the first section 4 being inserted into the incision.

FIG. 3 depicts an exploded view of the trocar 12 and first part 4. This enables the generally tubular configuration of the first part 4 to be seen. The first part 4 has an external surface which engages the incision and holds it open. An internal surface of the first part 4 then defines a single channel through the incision. The first part 4 has a flange 20 at its proximal end together with connection points 8′, 16′ for engaging corresponding connection points 8, 16 on the second part 6 and the trocar 12, respectively.

FIGS. 4 and 5 depict exploded views of the entire system according to this embodiment. The exploded views in FIGS. 4 and 5 show the trocar 12 which is used to insert the first part 4. However, once the first part 4 has been inserted the trocar 12 is removed and no longer required in the procedure.

As discussed above, the first part 4 has a generally tubular structure with a flange 20 at its proximal end. In this embodiment, the second part is assembled from a variety of other parts, to form a generally circular plate with several ports for surgical instruments connected to the plate via ball joints. The plate 22 has a diameter substantially equal to the diameter of the flange 20 of the first part. It also includes connection points 8 corresponding to connection points 8′ on the first part 4. In this embodiment the connection points 8 are formed by protrusions that are engaged in recesses on the first part 4.

Plate 22 has a proximal surface which defines three openings 24 (best seen in FIG. 5) for receiving the port assembly. Below the openings 24, the distal surface of the plate 22 defines a curved surface which is a portion of a hemisphere. This portion of a hemisphere combines with a lower plate 26 which defines openings 28 with curved surfaces that complete the hemisphere of the ball joint. As can be seen most clearly in FIG. 5 the openings 28 include a surface which defines a ridge 30 on its surface. In use, the ridge 30 provides improved sealing with the ball when the ball joint is assembled.

In FIGS. 4 and 5 the ports 32 are shown as a single assembly. However, they comprise two parts which are removably attached to each other. A lower part 34 comprises a ball with dimensions chosen such that it can be received in the generally spherical space formed when the plate 22 is adjacent lower plate 26. As can best be seen in FIG. 4 this ball 34 comprises a channel extending across its entire diameter, so that its lower, distal end is open. A reduced diameter neck portion 36 extends from the ball 34 and engages the port 38. In order to assemble the second part 6, the ball and neck 36 are removed from the port 38 and the neck 36 inserted through one of the openings 24 in the plate 8. The port 38 is then attached to the neck 36, for example with a screw or interference fit. The port assemblies 32 are then securely held in the plate 8.

The ports 38 have a proximal end adapted to receive valves 40. In this embodiment the valves 40 are “duckbill” valves, although any form of valve may be used in alternate embodiments. More generally, the valve may be a non-return valve incorporating a lip seal. The valves 40 may be inserted into the ports 38 by a press fit. In alternative embodiments, the valves 40 may be attached by unscrewing a retaining ring at the top of the port 38, placing the valve within the port and then reattaching the retaining ring.

The valves 40 include openings on their upper surface for receiving standard 5 mm diameter surgical instruments. In alternate embodiments, the different configurations of ports may be provided. For example, a different number of ports, such as four, two or five ports could be provided. The diameter of the surgical instruments received by the ports may also be varied, for example, two 5 mm ports may be provided in conjunction with a 10 mm port, or a 10, 12 and 5 mm port can be provided. Likewise, although in this embodiment all of the ports are connected by a ball joint, if relative movement between a surgical instrument and the apparatus 2 is not required, the ball joint may be omitted.

FIG. 6 depicts a cross-section of the apparatus 2 when the first part 4 and the second part 6 are attached. It shows how the interaction between the openings 24 in the plate 22 cooperate with the openings in the lower plate 26 to define a partially spherical joint for receiving ball 34 of the port. The passageway 44 extending the length of the port is also visible, as is the valve 40.

FIG. 6 shows how the valve 40 is located separate from the ball 34. This means that when the port is rotated relative to the apparatus 2, although the ball joint moves, the position of the valve relative to an inserted surgical instrument remains relatively constant, with the valve able to create a high quality seal due to the generally perpendicular arrangement between the valve faces and the inserted surgical instrument.

The first part 4 is preferably formed from an engineering plastic, such as PEEK. Likewise the plate 22 and ports 32 are also preferably formed from an engineering plastic, such as PEEK. The lower housing disc is advantageously flexible to improve sealing. It can be manufactured of an engineering plastic, such as PEEK or alternatively, a more flexible rubberised material, such as silicone rubber. The choice of material for lower plate 26 will depend on the interaction between the material of the lower plate 26 and the ball 34. The interaction between these materials will determine the coefficient of friction and relative stiffness between the two parts and must also provide reasonable sealing, although in this embodiment the fin 30 also improves the sealing.

The valves 40 are manufactured from silicone rubber or an alternative rubberised material. The trocar 12 is generally manufactured from stainless steel. However, parts of it, such as the central flange, may also be formed from an engineering plastic such as PEEK.

In the above embodiment, all of the components of the system are intended to be reusable apart from the lower plate 26 and the valves 40. This is because the sealing provided by the lower plate 26 and the valves 40 is improved by using a more flexible material, such as silicone rubber which may not be reusable. Because the second part 6 is intended to be reusable, once the ball ports 34 have been assembled in the plates 22, they will not generally be disassembled by a user. The second part 6 can be sterilised in its assembled form.

In an alternate embodiment, the valves 40 and lower plate 26 may also be reusable. Likewise any or all of the components may also be disposable in alternative embodiments.

Thus, the present invention provides an apparatus and system that allows simultaneous access for two or more surgical instruments through a single incision. The use of a ball joint enables flexible positioning of the surgical instrument relative to the apparatus without compromising sealing. The two part construction enables the apparatus to be installed easily using a trocar.

1. An apparatus for allowing simultaneous use of at least two surgical instruments through a single incision, the apparatus comprising:
a first part having a proximal end, a distal end, an inner surface and an outer surface, wherein the outer surface is for engaging an incision and holding the incision in an open position and the inner surface defines a passageway through the first part from the proximal to the distal ends; and
a second part attached to the proximal end of the first part for sealing the proximal end of the first part and comprising at least two ports for receiving a surgical instrument, and wherein at least one of the ports comprises a ball joint to allow articulation of the port relative to the second part.

2. An apparatus according to any claim 1, wherein each port further comprises a valve for sealing the surgical instrument, and wherein if the port comprises a ball joint, the valve is positioned separate from the ball joint.

3. An apparatus according to claim 1, wherein the ball joint comprises sealing fins located in the socket.

4. An apparatus according to claim 1, wherein the second part is releasably attached to the first part.

5. An apparatus according to claim 4, wherein the first part and/or the second part is reusable.

6. An apparatus according to claim 4, wherein the first part is adapted for insertion into an incision using a trocar.

7. A system comprising:
an apparatus according to claim 6; and
a trocar arranged to releasably attach to the proximal end of the first part, for inserting the first part into an incision.

8. An apparatus for allowing simultaneous use of at least two surgical instruments through a single incision, the apparatus comprising:
a first part having a proximal end, a distal end, an inner surface and an outer surface, wherein the outer surface is for engaging an incision and holding the incision in an open position and the inner surface defines a passageway through the first part from the proximal to the distal ends; and
a second part attached to the proximal end of the first part for sealing the proximal end of the first part and comprising at least two ports for receiving a surgical instrument, and wherein at least one of the ports comprises a ball joint to allow articulation of the port relative to the second part;
wherein the ball joint comprises a socket and the ball joint further comprises at least one ridge or sealing fin located within the socket.

9. An apparatus according to claim 8, wherein each port further comprises a valve for sealing the surgical instrument, and wherein if the port comprises a ball joint, the valve is positioned separate from the ball joint.

10. An apparatus according to claim 8, wherein the ball joint comprises sealing fins located in the socket.

11. An apparatus according to claim 8, wherein the second part is releasably attached to the first part.

12. An apparatus according to claim 11, wherein the first part and/or the second part is reusable.

13. An apparatus according to claim 11, wherein the first part is adapted for insertion into an incision using a trocar.

14. A system comprising:
an apparatus according to claim 13; and
a trocar arranged to releasably attach to the proximal end of the first part, for inserting the first part into an incision.
